(19) United States
(12) Patent Application Publication
(10) Pub. No.: US 2024/0415798 A1
(43) Pub. Date: Dec. 19, 2024

(54) COMPOSITIONS AND METHODS FOR TREATING MUCOSITIS

(71) Applicant: Enzychem Lifesciences Corporation, Seoul (KR)

(72) Inventors: Sun Young Yoon, Daejeon (KR); Ki Young Sohn, Seoul (KR)

(21) Appl. No.: 18/702,478

(22) PCT Filed: Oct. 18, 2022
(86) PCT No.: PCT/IB2022/060007

§ 371 Date: Apr. 18, 2024

Related U.S. Application Data
(60) Provisional application No. 63/257,099, filed on Oct. 18, 2021.

(51) Int. Cl. A61K 31/231; A61K 31/155; A61K 31/355; A61K 31/522; A61K 31/728; A61K 38/08; A61K 38/18; A61K 38/44; A61P 37/06

(52) U.S. Cl. CPC A61K 31/231; A61K 31/155; A61K 31/355; A61K 31/522; A61K 31/728; A61K 38/08; A61K 38/18; A61K 38/446; A61P 37/06

(57) ABSTRACT

In one aspect. methods are provided for treating a human subject having or susceptible to mucositis, comprising administering to the human subject an effective amount of EC-18, thereby treating or preventing the mucositis.

### CROSS REFERENCE TO RELATED APPLICATION(S)

The present application claims the benefit of U.S. provisional application 63/257,099 filed Oct. 18, 2021, which is incorporated by referenced herein in its entirety.

### FIELD

In one aspect, methods, compositions and kits are provided for treating, preventing or reducing the occurrence or severity of mucositis in human subjects. In a further aspect, methods, compositions and kits are provided for treating, preventing or reducing the occurrence or severity of oral mucositis in human subjects. In a yet further aspect, methods, compositions and kits are provided for treating, preventing or reducing the occurrence or severity of chemoradiation-induced oral mucositis (CRIOM) in human subjects. In a still further aspect, methods, compositions and kits are provided for treating, preventing or reducing the occurrence or severity of radiation-induced oral mucositis (RIOM).

### BACKGROUND

Oral mucositis is a frequent and severe adverse event in patients undergoing chemoradiotherapy including head and neck cancers. Occurrence may result in drop-out from treatment, thereby reducing patient survival.

Oral mucositis has been characterized by painful ulcerative lesions of the oral mucosa observed in patients with cancer, who are treated with chemotherapy, and/or with radiation therapy. Lalla et al., MASCC/ISOO clinical practice guidelines for the management of mucositis secondary to cancer therapy. Cancer. 2014; 120:1453-1461. doi: 10.1002/cncr.28592; Kashiwazaki et al. Professional oral health care reduces oral mucositis and febrile neutropenia in patients treated with allogeneic bone marrow transplantation. Support. Care Cancer. 2012;20:367-373. doi: 10.1007/s00520-011-1116-x.

The National Cancer Institute (NCI) published Common Terminology Criteria for Adverse Events (CTCAE). It includes separate subjective and objective scales for mucositis: Grade 1—Erythema of the mucosa; Grade 2—Patchy ulcerations or pseudomembranes; Grade 3—Confluent ulcerations or pseudomembranes; bleeding with minor trauma, Grade 4—Tissue necrosis; significant spontaneous bleeding; life-threatening consequences, Grade 5—Death.

Efforts have been made to address chemoradiation-induced oral mucositis (CRIOM) and radiation-induced oral mucositis (RIOM). See, for example, Liu et al. Status of Treatment and Prophylaxis for Radiation-Induced Oral Mucositis in Patients With Head and Neck Cancer. Front. Oncol. 11:642575. doi: 10.3389/fonc.2021.642575. Current clinical approaches often have not been satisfactory.

It would be desirable have new therapies for treating, preventing or reducing the occurrence or severity of mucositis.

### SUMMARY

We have now found that the compound EC-18 is effective in treating human subject that suffer or are susceptible to oral mucositis.

In particular, we have found that EC-18 is effective in treating human subjects that suffer or are susceptible to oral mucositis as occurring during the course of a chemo-radiation therapy for treatment of head or neck cancer.

EC-18 treatment for the present indications has been shown to effectively occur in a dose dependent manner.

In one embodiment, the mucositis may be oral, gastrointestinal, or alimentary mucositis. In another embodiment, the mucositis may be associated with cancer, chemotherapy, radiotherapy, or the combination of chemotherapy and radiotherapy.

In one aspect, methods are provided for treating severe mucositis including severe oral mucositis (SOM), which may include symptoms of extensive oral ulcerations.

EC-18 may be represented by a structure of the following Formula 1:

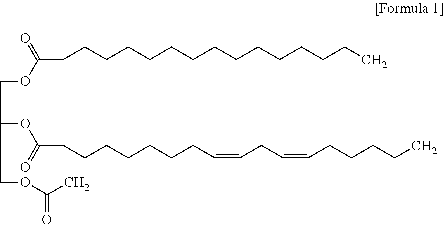 [SMILES: CCCCC/C=C\C/C=C\CCCCCCCC(=O)OC(COC(C)=O)COC(=O)CCCCCCCCCCCCCCC]

The compound of Formula 1 is sometimes referred as “PLAG” or “EC-18” in this disclosure.

As discussed, EC-18 may be used to treat a human subject suffering from or susceptible to mucositis, including oral mucositis such as may be associated with cancer, chemotherapy, radiotherapy, the combination of chemotherapy and radiotherapy. In a further embodiment,. Also, EC-18 may be used to treat a human subject suffering from or susceptible to mucositis (e.g., oral mucositis) in a human subject having or susceptible to mucositis in head and neck cancer. Also, EC-18 may be used to treat a human subject suffering from or susceptible to mucositis (e.g., oral mucositis) in a human subject having or susceptible to mucositis in head and neck cancer associated with a human papillomavirus (HPV) or its infection.

In certain preferred aspects. a human patient may receive multiple doses of EC-18 per day

In certain preferred aspects, a human patient may receive EC-18 in oral form such as capsule or tablet.

In certain preferred aspects, a human patient may receive EC-18 in daily dosages of up to 500 mg, or up to 600, 700, 800, 900 or 1000 mg EC-18 per day, or up to 1100, 1200,1300, 1400, 1500 mg EC-18 per day, or up to 1600, 1700, 180, 199 or 2000 mg EC-18 per day, or up to 2100, 2200, 2300, 2400, 2500 mg EC-18 per day, or high doses such as 2500,300, 3500 or 4,000 or more mg EC-18 per day

A human subject may be determined to have or be susceptible for treatment by undergoing chemical or radiation cancer therapy. Such subjects may display for example oral ulceration.

In particular aspects, a human subject will be identified and selected for treatment as disclosed herein, and then EC-18 will be administered to the identified and selected subject. In particular, a human patient may be identified as suffering or susceptible to oral mucositis and the identified patient will be selected for treatment and an effective amount of EC-18 will be administered to the identified and selected patient. A human patient also may be identified as suffering or susceptible to oral mucositis as well as being HPV positive and the identified patient will be selected for treatment and an effective amount of EC-18 will be administered to the identified and selected patient.

In certain aspects, EC-18 may be administered to a subject in combination or coordination with one or more therapeutic agents that are distinct from EC-18. For instance, EC-18 may be administered in combination with palifermin; pentoxifylline including with vitamin E; chlorhexidine gluconate (including oral rinse); hyaluronate including sodium hyaluronate; a superoxide dismutase mimetic such as superoxide dismutase mimetic M40403; and/or dusquetide or other innate defense regulator.

In a further aspect, pharmaceutical compositions are provided comprising EC-18. The compositions suitably may comprise one or more pharmaceutically acceptable carriers. In preferred embodiments, the compositions may be formulated or otherwise adapted for treatment of mucositis as disclosed herein. In preferred aspects, the composition may be adapted for oral administration such as a tablet or capsule.

In a yet further aspect, kits are provided for use to treat or prevent mucositis as disclosed herein. Kits of the invention suitably may comprise 1) a therapeutically effective amount of EC-18; and 2) instructions for using the EC-18 for treating or preventing mucositis. The instructions suitably may be in written form, including as a product label.

The human subject for treatment may be a male or female. In certain aspects, the human will be an adult (at least 16, 18 or 21 years of age). In certain aspects, the human will be a pediatric patient (less than 18, 16, 14, 12, 10, 8 or 6 years of age).

In one embodiment, a method of treating or preventing oral mucositis associated with chemotherapy and/or radiotherapy may comprise administration of a composition comprising an effective amount of EC-18 to a human subject. In another embodiment, a method of treating or preventing alimentary tract mucositis such as may be associated with chemotherapy and/or radiotherapy may comprise administration of a comprising an effective amount of EC-18 to a human subject. In another embodiment, a method of treating or preventing gastrointestinal tract mucositis associated with chemotherapy and/or radiotherapy may comprise administration of a composition comprising an effective amount of EC-18 to a human subject.

In one embodiment, the method of treating or preventing mucositis associated with cancer may comprise administration of a composition comprising an effective amount of an EC-18 to a human subject. In another embodiment, a method of treating or preventing oral mucositis associated with cancer may comprise administration of a composition comprising an effective amount of EC-18 to a human subject. In another embodiment, a method of treating or preventing alimentary tract mucositis associated with cancer may comprise administration of a composition comprising an effective amount of EC-18 to a human subject. In another embodiment, a method of treating or preventing gastrointestinal tract mucositis associated with cancer may comprise administration of a composition comprising an effective amount of EC-18 to a human subject.

In one embodiment, the method of treating or preventing mucositis associated with hematopoictic stem cell transplant (HSCT) may comprise administration of a composition comprising an effective amount of EC-18 to a human subject. In another embodiment, a method of treating or preventing oral mucositis associated with hematopoietic stem cell transplant (HSCT) may comprise administration of a composition comprising an effective amount of EC-18 to a human subject. In another embodiment, a method of treating or preventing alimentary tract mucositis associated with hematopoietic stem cell transplant (HSCT) may comprise administration of a composition comprising an effective amount of EC-18 to a human subject. In another embodiment, a method of treating or preventing gastrointestinal tract mucositis associated with hematopoietic stem cell transplant (HSCT) may comprise administration of a composition comprising an effective amount of EC-18 to a human subject

A subject may have mucositis (e.g., oral mucositis or oral stomatitis) or may be at risk of developing mucositis (for example, a patient who is receiving or who is about to receive chemotherapy and/or radiation therapy). By way of additional example, a subject in need may be a female subject who has AV (atrophic vaginitis) or vaginal mucositis or who is at risk of developing vaginal mucositis or AV (e.g., a woman who presents symptoms that may precede clinical manifestation of AV, such as vaginal dryness).

In one embodiment, the method of treating or preventing mucositis associated with hematopoietic stem cell transplant (HSCT) may comprise administration of a composition comprising an effective amount of EC-18 to a human subject.

In one aspect, the subject may have a human papillomavirus (HPV) infection. In one aspect, the subject may test positive for human papillomavirus (HPV), for example a positive HPV test via a cervical test. We have surprisingly found that human subjects being treated for mucositis as disclosed herein who are also HPV positive exhibit particularly favorable therapeutic benefits from the EC-18 treatment.

In further aspects, use of EC-18 is provided including to treat a human subject suffering from or susceptible to a condition as disclosed herein, particularly oral mucositis as occurring during the course of a chemo-radiation therapy for treatment of head or neck cancer. In additional aspects, use of EC-18 is provided to prepare a medicament including to treat a human subject suffering from or susceptible to a condition as disclosed herein, particularly oral mucositis as occurring during the course of a chemo-radiation therapy for treatment of head or neck cancer.

Other aspects of the invention are disclosed infra.

### BRIEF DESCRIPTION OF DRAWING

The patent or application file contains at least one drawing executed in color. Copies of this patent or patent application publication with color drawings will be provided by the Office upon request and payment of the necessary fee.

FIG. 1 shows patients flowchart for both stages 1 and 2 with the definition of each patient population. To truly observe and analyze the efficacy analysis of EC-18 with the comparison of placebo, PP (per protocol) up to STFU (short-term follow-up) period (approximately 4-6 weeks after a 7-week active treatment period) was analyzed. The efficacy results addressed in the contents are based on placebo (Stage 1 n=2, Stage 2 n=18:Total: 20) and EC-18 group (Stage 1 n=5, Stage 2 n=17: Total: 22)

FIGS. 2A-2B shows duration of severe oral mucositis (SOM) at Primary endpoint. In the PP population (population used for supportive efficacy analyses), the median duration of SOM from baseline through the STFU period was shorter in EC-18 2000 mg group as compared with Placebo (0.0 days vs 13.5 days)

FIGS. 3A-3B shows incidence of SOM at secondary efficacy endpoints. In the PP population, the incidence of SOM from baseline through the active treatment period and STFU were reported less in EC-18 2000 mg group as compared with Placebo group (10[45.5%] vs 14 [70.0%]

FIG. 4 shows cumulative SOM incidence at each week interval over 7 weeks of treatment. In the PP population, the incidence of SOM from baseline through the active treatment period was reported less in EC-18 2,000 mg group as compared with Placebo group [(36.4%) vs (65.0%) subjects]

FIGS. 5A-5B shows time to SOM onset at the secondary endpoints.

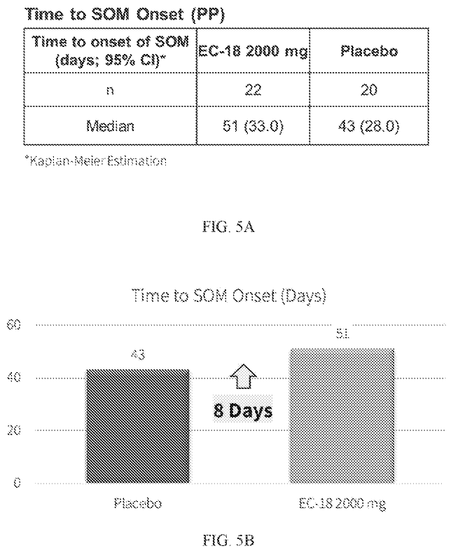

FIG. 6 (includes FIGS. 6A and 6B) shows time for Opioid use at the secondary endpoints.

FIG. 7 shows covariate analysis based on Cisplatin regimen and HPV status.

FIG. 8 shows TGFβ level comparison between EC-18 and Placebo groups in total.

FIGS. 9A-9B show TGFβ comparison between EC-18 and Placebo groups with SOM (FIG. 9A) and without SOM (FIG. 9B).

FIGS. 10A-10B show TGFβ level comparison between EC-18 and Placebo groups with HPV+ (FIG. 10A) and without HPV (FIG. 10B).

FIGS. 11A-11B show TGFβ level comparison between EC-18 and Placebo groups treated with cisplatin weekly (FIG. 11A) and cisplatin tri-weekly (FIG. 11B).

FIG. 12 shows IL-1β level comparison between EC-18 and Placebo groups in total.

FIGS. 13A-13B show IL-1β comparison between EC-18 and Placebo groups with SOM (FIG. 13A) and without SOM (FIG. 13B)

FIGS. 14A-14B show IL-1β level comparison between EC-18 and Placebo groups with HPV+ (FIG. 14A) and HPV− (FIG. 14B).

FIGS. 15A-15B show IL-1β level comparison between EC-18 and Placebo groups with cisplatin weekly (FIG. 15A) and cisplatin tri-weekly (FIG. 15B).

### DETAILED DESCRIPTION

We have now demonstrated in human clinical trials that EC-18 is highly effective to treat, reduce the severity and otherwise ameliorate mucositis in patients who have received chemotherapy and/or radiation therapy for treatment of cancer.

In one aspect, a human subject receiving treatment accordance with the present methods may be suffering from one or more types of cancer, including a solid tumor such as may be associated with head or neck cancer. The human subject may be receiving chemotherapy for the cancer, such as with cyclophosphamide, doxorubicin, etoposide, ifosfamide, mesna, cisplatin, gemcitabine and/or tamoxifen, or one or more other chemotherapeutic agents.

### Combination Therapy

As discussed, EC-18 can be administered in combination such as to treat a subject including a human suffering from or susceptible to mucositis, or suffering from symptoms of mucositis. The one or more distinct therapeutic agents that can be administered in combination include for example palifermin: pentoxifylline including with vitamin E; chlorhexidine gluconate (including oral rinse); hyaluronate including sodium hyaluronate; a superoxide dismutase mimetic such as superoxide dismutase mimetic M40403; dusquetide or other innate defense regulator.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1minute, 5 minutes, 15 minutes, 30 minutes, 45 minutes, 1 hour, 2 hours, 4 hours, 6 hours, 12hours, 24 hours, 48 hours, 72 hours, 96 hours, 1 week, 2 weeks, 3 weeks, 4 weeks, 5 weeks, 6weeks, 8 weeks, or 12 weeks before), concomitantly with, or subsequent to (e.g., 1 minute, 5minutes, 15 minutes, 30 minutes, 45 minutes, 1 hour, 2 hours, 4 hours, 6 hours, 12 hours, 24hours, 48 hours, 72 hours, 96 hours, 1 week, 2 weeks, 3 weeks, 4 weeks, 5 weeks, 6 weeks, 8wecks, or 12 weeks after) the administration of a second therapy to a subject in need of treatment as disclosed herein. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

The administration of EC-18 and the one or more distinct therapeutic agents may be by suitable means that results in a concentration of the therapeutic that, combined with other components, is effective in ameliorating, reducing, or stabilizing symptoms of mucositis including the occurrence of symptoms of mucositis.

The EC-18 and the one or more distinct therapeutic agents may be administered simultaneously or sequentially. In some embodiments, the one or more distinct therapeutic agents have been reported for use to treat mucositis.

As discussed, 1) EC-18 and 2) one or more distinct therapeutic agents may be “co-administered”, i.e, administered together in a coordinated fashion to a subject, either as separate pharmaceutical compositions or admixed in a single pharmaceutical composition. By “co-administered”, the one or more additional distinct therapeutic agents may also be administered simultaneously with EC-18, or be administered sequentially or separately (e.g. each agent being administered at least 5, 10, 30, 60, 120, 180, 240, 300, 360, 420 or 500 minutes apart) with EC-18, including at different times and with different frequencies. The one or more distinct therapeutic agents may be administered by any appropriate route for the agent(s), such as orally, intravenously, subcutaneously, intramuscularly, nasally, and the like; and the therapeutic agent may also be administered by any conventional route. In at least certain embodiments, at the one or more distinct therapeutic agents may be administered orally.

In some embodiments, EC-18 and/or the one or more distinct therapeutic agents may be administered daily, e.g . . . every 24 hours, or, continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Exemplary effective daily doses of the distinct therapeutic agent(s) include between 0.1 μg/kg and 100 μg/kg body weight, e.g., 0.1, 0.3. 0.5, 1, 5, 10, 15, 20, 25, 30, 35, 40, 45,50, 55, 60, 65, 70, 75, 80, 85, 90, 95, or 99 μg/kg body weight.

Alternatively, the distinct therapeutic agent(s) are administered about once per week, e.g., about once every 7 days. Or, the distinct therapeutic agent(s) are administered twice per week, three times per week, four times per week, five times per week, six times per week, or seven times per week. Exemplary effective weekly doses of the distinct therapeutic agent(s) include between 0.0001 mg/kg and 4 mg/kg body weight, e.g., 0.001, 0.003, 0.005, 0.01,0.02, 0.03, 0.04, 0.05, 0.06, 0.07, 0.08, 0.09, 0.1, 0.2, 0.3. 0.4, 0.5, 0.6, 0.7, 0.8, 0.9, 1, 2, 3, or 4 mg/kg body weight. For example, an effective weekly dose of the distinct therapeutic agent(s) is between 0.1 μg/kg body weight and 400 μg/kg body weight.

### Compositions and Treatments

The pharmaceutical composition comprising EC-18 may include conventional pharmaceutically acceptable carriers, excipients, or diluents. The amount of EC-18 can be widely varied without specific limitation, and is specifically 0.0001 to 100 weight %. preferably, 0.001 to 90 weight %, for example, EC-18 may be contained in 70 to 80 weight %, with respect to the total amount of the composition.

Suitable dosages of EC-18 may suitably vary. Preferably a dosage is provided that alleviates mucositis or reduces the likelihood of occurrence of mucositis in a human subject. An exemplary daily dosages for a human patient in need of treatment of EC-18 include between 0.0001 mg/kg and 4 mg/kg body weight, or between 0.01 mg/kg and 4 mg/kg body weight e.g., up to or about 0.001, 0.003, 0.005, 0.01, 0.02, 0.03, 0.04, 0.05, 0.06, 0.07, 0.08,0.09, 0.1, 0.2, 0.3, 0.4, 0.5, 0.6, 0.7, 0.8, 0.9, 1, 2, 3, 4, 5, 10, 20, 30, 40, 50, 60, 70, 80, 90 or 100 mg/kg body weight of the human subject. For example, in some embodiments, an effective weekly dose of EC-18 may between 0.1 μg/kg body weight and 400 μg/kg body weight of the human patient in need thereof. In preferred aspects, an oral formulation is utilized such as a tablet or capsule (e.g. soft gelatin capsule) that contains 250-1000 mg, e.g., 500 mg, of EC-18. Optimal dosage amounts also can be determined empirically for particular patients or identified group of patients (e.g. other one or more other pre-existing conditions, for instance, patients with history of hypertension; patients with cancer; patients with HPV or its infection).

The pharmaceutical composition of the present invention may further include other active ingredients having a therapeutic effect. The pharmaceutical composition may be formulated into solid, liquid, gel or suspension form for oral or non-oral administration, for example, tablet, bolus, powder, granule, capsule such as hard or soft gelatin capsule, emulsion, suspension, syrup, emulsifiable concentrate, sterilized aqueous solution, non-aqueous solution, freeze-dried formulation, and so on. In formulating the composition, conventional excipients or diluents such as fillers, bulking agents, binders, wetting agents, disintegrating agents, and surfactants can be used. The solid formulation for oral administration includes tablet, bolus, powder, granule, capsule and so on, and the solid formulation can be prepared by mixing one or more of the active components and at least one excipient such as starch, calcium carbonate, sucrose, lactose, gelatin, and so on. Besides the excipient, a lubricant such as Magnesium stearate and tale can also be used. The liquid formulation for oral administration includes emulsion, suspension, syrup, and so on, and may include conventional diluents such as water and liquid paraffin or may include various excipients such as wetting agents, sweeting agents, flavoring agents, and preserving agents. The formulation for non-oral administration includes sterilized aqueous solution, non-aqueous solution, freeze-dried formulation, suppository, and so on, and solvent for such solution may include propylene glycol, polyethylene glycol, vegetable oils such as olive oil, and ester for syringe injection such as ethyl oleate. Base materials of the suppository may include witepsol, macrogol, tween 61, cacao butter, Laurin and glycerogelatin.

EC-18 can be administered in a pharmaceutically effective amount. The term “pharmaceutically effective amount” is used to refer to an amount that is sufficient to achieve a desired result in a medical treatment. The “pharmaceutically effective amount” can be determined according to the subject's category, age, sex, severity and type of disease, activity of drug, sensitivity to drug, administration time, administration route, excretion rate, and so forth. The composition of the present invention can be administered alone or with other therapeutic agents sequentially or simultaneously. The composition of the present invention can be administered once or multiple times. The preferable amount of the composition of the present invention can be varied according to the condition and weight of patient, severity of disease, formulation type of drug, administration route and period of treatment. An appropriate total amount of administration per 1 day can be determined by a physician and is generally about 0.001 to about 5,000 mg/kg, preferably about 0.05 to 1,000 mg/kg, twice a day or can be administered in divided doses multiple times daily.

The term “administration” means introducing the pharmaceutical composition of the present invention to a patient in need by any suitable method. The route of administration may be any or a various routes, oral or non-oral, as long as the target tissue can be reached, for example, oral administration, intraperitoneal administration, transdermal administration (topical application, etc.), intravenous administration, intramuscular administration, subcutaneous administration, intradermal administration, intranasal administration, rectal administration, intranasal administration, intraperitoneal administration and the like may be used, but is not limited thereto. Oral administration is preferred.

As discussed, in the present therapies, EC-18 is administered to a human subject suffering from or susceptible to mucositis, including oral mucositis such as may be associated with cancer, chemotherapy, radiotherapy, the combination of chemotherapy and radiotherapy. In a particular therapy, EC-18 is administered to a human subject suffering from or susceptible to oral mucositis that has head or neck cancer.

Also, EC-18 may be used to treat a human subject suffering from or susceptible to mucositis (e.g., oral mucositis) in a human subject having or susceptible to mucositis in head and neck cancer associated with a human papillomavirus (HPV) or its infection.

As discussed, the human subject being treated may have a human papillomavirus (HPV) infection. In one aspect, the subject may test positive for human papillomavirus (HPV), for example a positive HPV test via a cervical test. See the results set forth in the Examples which follow, which demonstrate effective EC-18 treatment of HPV positive human subjects.

A human subject may be determined to have or be susceptible for treatment by undergoing chemical or radiation cancer therapy. Such subjects may display for example oral ulceration

As discussed, a human subject may be identified and selected for treatment as disclosed herein, and then EC-18 will be administered to the identified and selected subject. For instance, a human patient may be identified as suffering or susceptible to oral mucositis and the identified patient will be selected for treatment and an effective amount of EC-18 will be administered to the identified and selected patient. A human patient also may be identified as suffering or susceptible to oral mucositis as well as being HPV positive and the identified patient will be selected for treatment and an effective amount of EC-18 will be administered to the identified and selected patient.

A subject may have mucositis (e.g., oral mucositis or oral stomatitis) or may be at risk of developing mucositis (for example, a patient who is receiving or who is about to receive chemotherapy and/or radiation therapy).

In one embodiment, the method of treating or preventing mucositis associated with hematopoietic stem cell transplant (HSCT) may comprise administration of a composition comprising an effective amount of EC-18 to a human subject.

In a yet further aspect, kits are provided for use to treat or prevent mucositis as disclosed herein. Kits of the invention suitably may comprise 1) a therapeutically effective amount of EC-18; and 2) instructions for using the EC-18 for treating or preventing mucositis. The instructions suitably may be in written form, including as a product label.

### EXAMPLES

The following examples are illustrative.

### Example 1: Patient Group and Clinical Study

Human patients of the following characteristics were enrolled:
18 years or older
Diagnosis of squamous cell carcinoma of the mouth, oropharynx, hypopharynx or nasopharynx
Planned to receive IMRT with daily fractions of 2.0 Gy to 2.2 Gy to a cumulative dose of at least 60 Gy and a maximum of 72 Gy
Radiation fields to include at least two mucositis sites at risk (buccal mucosa, floor of mouth, ventral and lateral tongue, soft palate) in which both sites receive a minimum cumulative dose of 55 Gy
Planned to receive concomitant single agent chemotherapy with cisplatin

EC-18 was administered in varying patients of the treatment group up to 2000 mg/day. Recommended therapy was 500 mg EC-18 capsule administered 4 times throughout the day (e.g., 2 capsules of 500 mg EC-18 administered within 30 mins after taking breakfast and dinner) for a total daily dosage of 2000 mg EC-18 per day. Treatment protocol lasted 7 weeks while radiation therapy and cisplatin therapy continued during the same time.

The EC-18 treatment group demonstrated clear therapeutics benefit over the control group, including reduction of mucositis symptoms.

For the study, 25 large institutions and hospitals experienced with head and neck cancer treatment were selected
Stage 1: To Evaluate safety and MTD that can be used for Stage 2 iDSMB: Data and Safety Monitoring Board reviewed safety data every 2 weeks and approved for Stage 2 using MTD 2000 mg.
Stage 2: To Evaluate safety and efficacy 7 weeks Active and 4-6 weeks of STFU until OM is resolved.
2.0-2.2 Gy daily fraction (5 days a week for 7 weeks) and I week (40 mg/m2) or every 3 weeks (100 mg/m2) cisplatin.
Minimum cumulative radiation dose of 55 Gy.

For Efficacy, measured duration of severe oral mucositis (SOM), Incidence of SOM during active treatment.

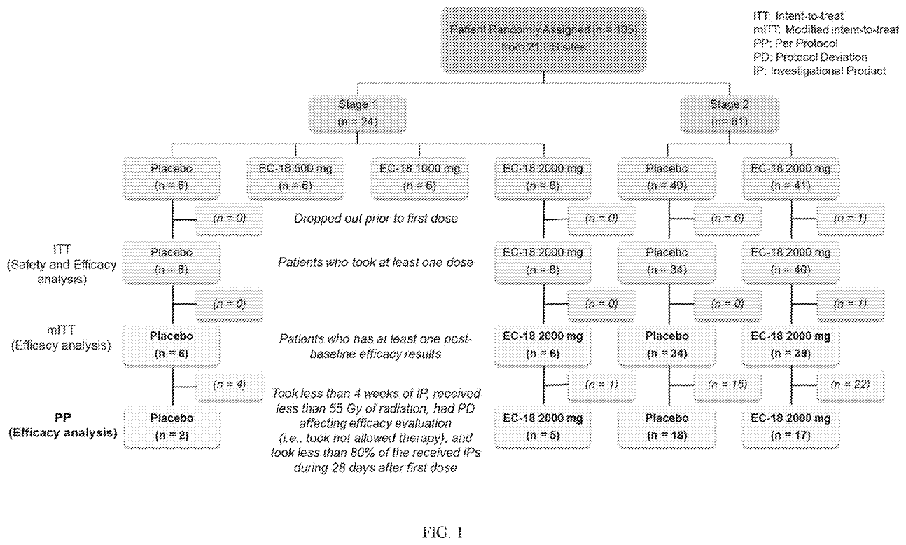

FIG. 1 (flow diagram) shows patient pool at the time of study start and factors leading to efficacy analysis population.

In Stage 1, we included Stage 1 placebo and 2000 mg patients to Stage 2 for efficacy analysis:
Placebo group: Stage 1 n=2, Stage 2 n=18: Total: 20
EC-18 2000 mg group: Stage 1 n=5, Stage 2 n=17: Total: 22

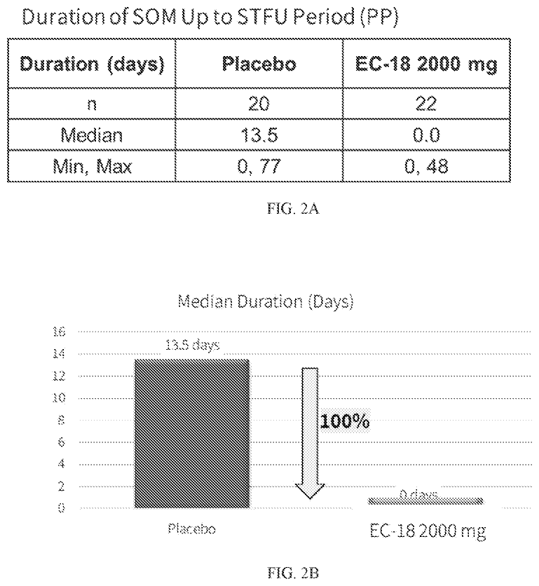

FIGS. 2A-2B summarize efficacy results. When measured Median of Duration and Incidence of SOM from the time when SOM develops to when SOM has been resolved; we see very strong efficacy results. With placebo group, we calculated median Duration of SOM almost 14 days compared to EC-18 group which never developed SOM.

This placebo group duration of SOM of 14 days is pretty close to what other agents have reported which indicated we are analyzing the correct population.

Similarly for Incidence of SOM for placebo population, many joumals and articles already reported that SOM will occur anywhere from 65% to 70%. Compared to placebo incidence of SOM, the EC-18 group incidence of SOM is at 45% which is over 35% reduction when compared to placebo population.

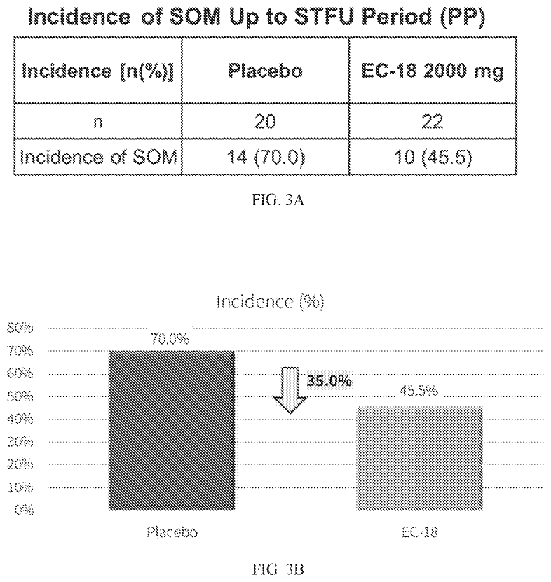

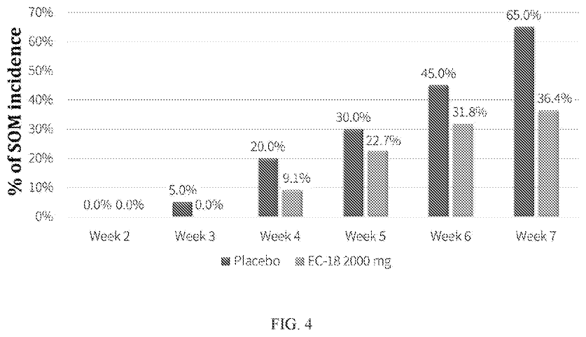

FIGS. 3A-3B graph shows percentage of SOM incidence at each week interval. SOM does not begin until week 3 and peaks at week 7 (by then, cumulative radiation dose is 70 Gy) and even at week 8 which is 1 week after the last radiation treatment. As can be seen in FIGS. 3A-3B, SOM incidence for EC-18 group is less than placebo group (PP Population or PP). As shown in FIG. 4, oral mucositis (OM) rarely develops after 1 dose of radiation and SOM was seen as cumulative dose increase (after week 3 or 4 and thereafter) and some extends beyond 7 weeks. In FIG. 4, the EC-18-treated group is shown on the right in each time period (each of Weeks 4, 5, 6 and 7), and the placebo shown on the left for each time period or alone in Week 3.

FIGS. SA-SB also show time to SOM onset at the secondary endpoints. In the PP population, the SOM onset was 8 days delayed in EC-18, 2,000 mg group compared with placebo group (51 days vs. 43 days).

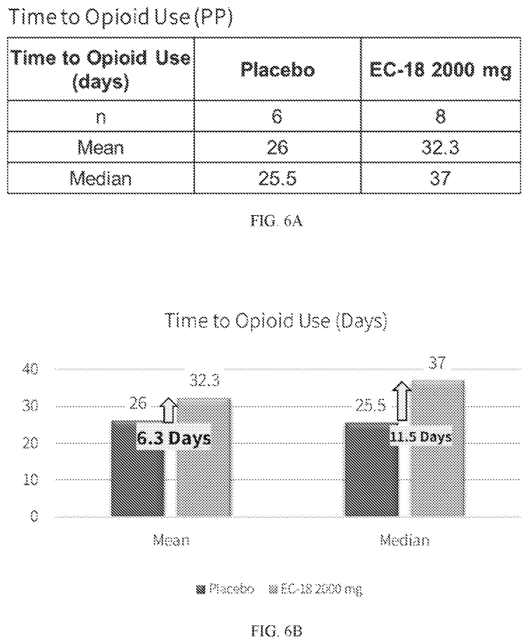

FIG. 6 (includes FIGS. 6A and 6B) shows time for Opioid use at the secondary endpoints. In the PP population, the time to first use of opioid analgesics was delayed for evaluable subjects (those who were not taking opioids analgesic immediately prior to study start or at baseline and those for whom data was available) among EC-18 2000 mg group (n=8) vs. Placebo group (n=6). The mean value of 6.3 days was delayed in EC-18 2,000 mg group compared with placebo group (32.3 days in EC-18 groups vs. 26 days in placebo). The median value of 11.5 days was delayed in EC-18 2,000 mg group compared with the placebo group (37 days in EC-18 groups vs. 25.5 days in placebo). In FIG. 6B, in each time period (6.3 days or 11.5 days), the placebo is shown on the left (26 at 6.3 days and 25.5 at 11.5 days) and the EC-18 2000 mg is shown on the right (32.3 at 6.3 days an 37 at 11.5 days).

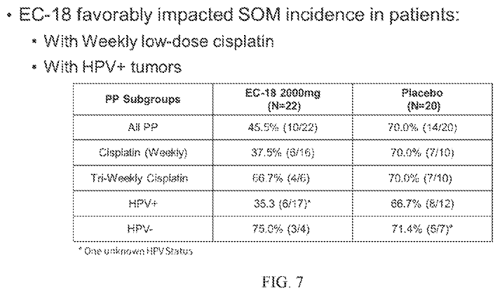

FIG. 7 shows covariate analysis based on Cisplatin regimen and HPV status. The observed results for incidence of SOM in subjects were less in the EC-18 2000 mg group as compared to the Placebo group with weekly cisplatin (37.5% vs. 70.0%), and with tri-weekly cisplatin (66.7% and 70.0%). respectively. The observed results for incidence of SOM in subjects with HPV+ were less in the EC-18 2000 mg group as compared to the Placebo group (35.3% vs. 66.7%). Whereas, there was no difference observed in the subjects with HPV− in the EC-18 2000 mg group as compared to the Placebo group (75% vs. 71.4%).

Safety results (e.g., serious adverse event (SAE)) shows no appreciable differences in SAEs were observed across the study arms. Moreover, all the reported AEs were attributable to chemoradiation treatment and not to EC-18. Stage 1 had 17 total SAEs from a total of 24 patients when Stage 2 had 31 total SAEs from a total of 81 patients. 020-018 patient was determined by investigator to be not related to study drug. Many SAEs occurred due to nausea and vomiting which is a normal symptom for chemo treatment

### Example 2: Cytokine profile From Patient Plasma samples in CRIOM Phase 2

### Assay Information: Luminex Assay

The plasma sample collected from the blood of patients listed in (Table 1 below) was thawed and centrifuged at 10,000 rpm at 4° C. for 2 minutes, and the supernatant was used for analysis. The supernatant was serially diluted, and 50 μl of the samples were dispensed to each well in a 96-well plate. 5O μl of diluted Microparticle Cocktail (R&D systems) shown in Table 2 was added to each well containing samples and incubated for 2 hours at room temperature on a shaker at 800 rpm. The sample mixture was removed and washed with 100 μl of washing buffer three times. Then, 50 μL of diluted Biotin-Antibody Cocktail (R&D systems) to each well and incubate for 1 hour at RT on the shaker at 800 rpm, followed by three times of washing with wash buffer. 50 μL of diluted Biotin-Antibody Cocktail (R&D systems) was added to each well and incubated for 30 minutes at RT on the shaker at 800 rpm. After washing with wash buffer three times, 100 μl of wash buffer was added to each well, and the sample's fluorescence intensity was obtained using a Luminex analyzer (Luminex) within 90 mins.

Standard, control and the sample was quantitatively analyzed using the average value of the results obtained by duplicate test.

The absolute value of each sample's mean fluorescence intensity (MFI) was obtained by comparing it to the standard curve. The value derived as an under-standard was marked as 0 because it is a trace value outside the detection range of the assay kit.

The standard curve was obtained by the best fit method in ‘MasterPlex QT 2010 (MiraiBio, Hitachi, CA, USA)’ to calculate the response measured value (MFI) for each standard concentration, which has the highest R square value among each calculation method (Highest value =1.0). The sample's resultant concentration value was calculated based on this standard curve.

TABLE 1
Sample information

| Sample name | CRIOM phase II patient plasma sample | | | | | |
|---|---|---|---|---|---|---|
| Sample | A group of patients who completed chemoradiotherapy and | | | | | |
| condition | substance administration according to the clinical trial plan | | | | | |
| | Total count: 96 | | | | | |
| | Number of samples in the analysis group | | | | | |
| | | Subject | Week 1 | Week 3/4 | Week 7 | Total |
| Sample | Placebo | 14 | 14 | 23 | 11 | 48 |
| available | Active | 14 | 14 | 21 | 13 | 48 |
| | (EC-18) | | | | | |

TABLE 2
Luminex assay kit information

| Target | Assay type | Dilution |
|---|---|---|
| TGFβ | Multiplex | 1:15 |
| IL-1β | Multiplex | 1:2 |

### TGF-β Level Comparison Between EC-18 and Placebo Groups in Total

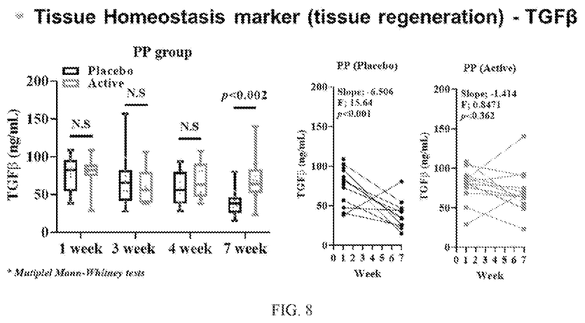

The TGF-β concentration in the EC-18 treated group was significantly higher than the placebo group at the 7^{th} week of chemoradiation therapy treatment. As show in FIG. 8, The TGF-β concentration in the EC-18 treated group was significantly higher than the placebo group at the 7th week of chemoradiation therapy treatment. In FIG. 8, in the graph on the left, the EC-18-treated group (active) is shown on the right in each time period, and the placebo shown on the left for each time period.

TGFβ Comparison Between EC-18 and Placebo Groups with SOM or without SOM

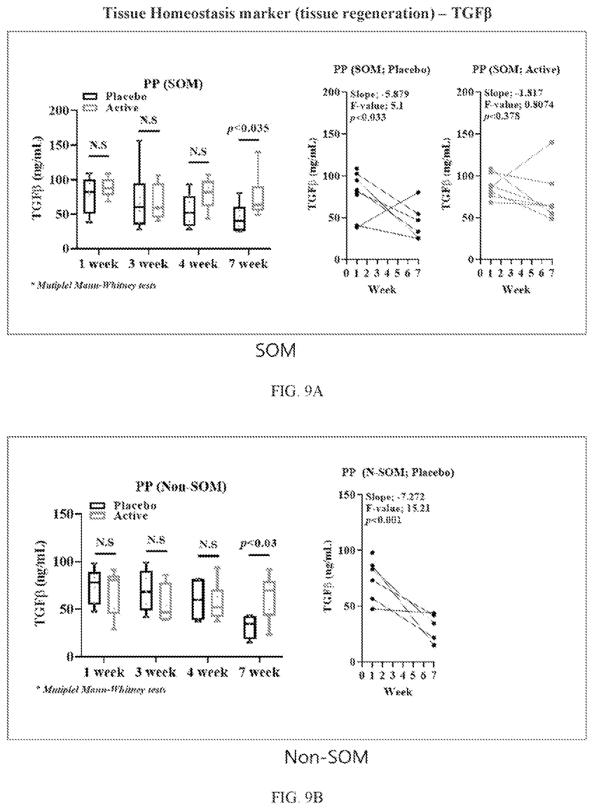

The TGF-β concentration in the EC-18 treated group was higher compared with the placebo group regardless of the incidence of SOM. As shown in FIGS. 9A-9B, the TGF-β concentration in the EC-18 treated group was higher compared with the placebo group regardless of the incidence of SOM. In FIGS, 9A and 9B, in the graph on the left, the EC-18-treated group (active) is shown on the right in each time period, and the placebo shown on the left for each time period.

TGFβ Level Comparison Between EC-18 and Placebo Groups with HPV+/HPV−

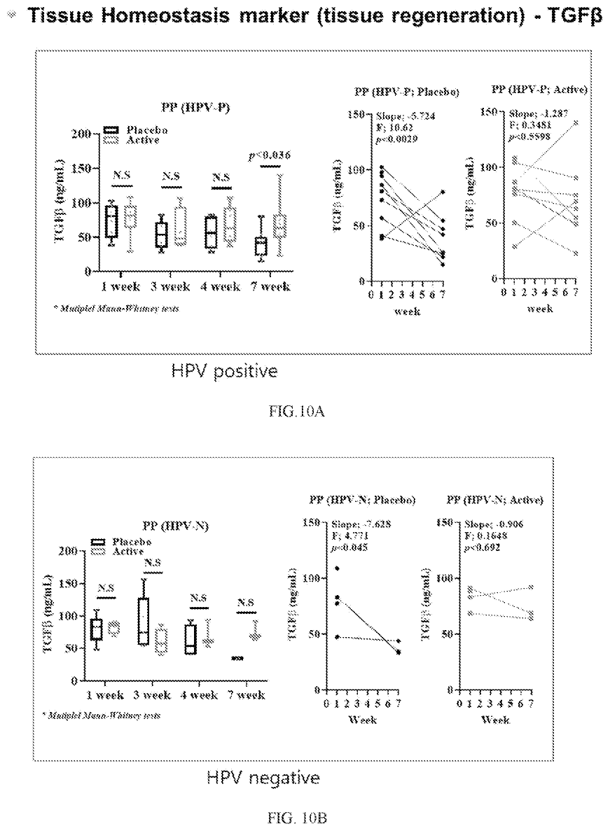

In the population of HPV-positive, the TGF-β concentration in the EC-18 was significantly higher compared with placebo. Although the HPV-negative patients were too small to conclude the statistical significance of the difference of TGFβ, the TGFβ level in the EC-18 group was similar to that in the placebo. As shown in FIGS. 10A-10B, in the population of HPV-positive, the TGF-β concentration in the EC-18 was significantly higher compared with the placebo. In FIGS, 10A and 10B, in the graph on the left, the EC-18-treated group (active) is shown on the right in each time period, and the placebo shown on the left for each time period.

TGFβ Level Comparison Between EC-18 and Placebo Groups Treated with Cisplatin Weekly or Cisplatin Tri-Weekly

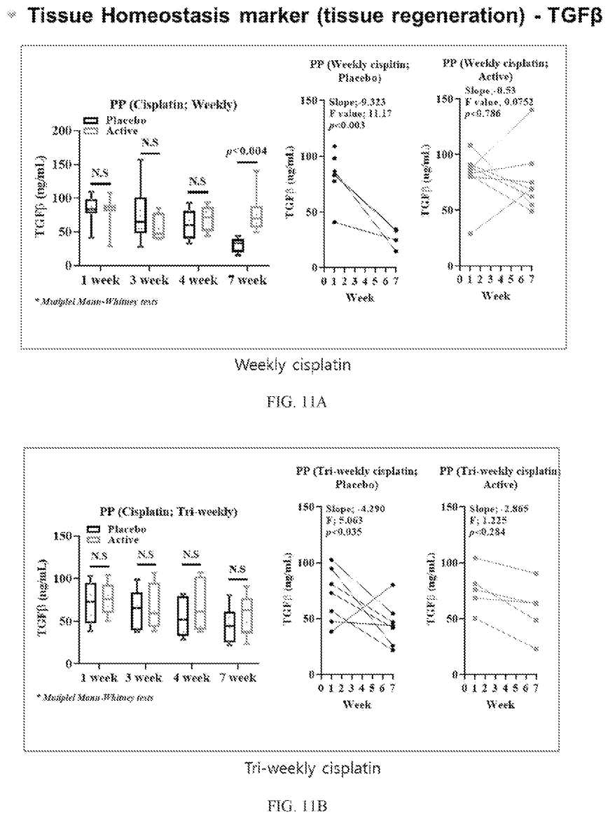

In the sub-population treated with cisplatin once a week, the TGF-β concentration in the EC-18 was significantly higher compared with placebo. However, TGFβ concentration was not significantly different between EC-18 group and placebo group treated with tri-weekly cisplatin. As shown in FIGS. 11A-11B, in the sub-population treated with cisplatin once a week, the TGF-β concentration in the EC-18 was significantly higher compared with placebo, but not in the EC-18 group with tri-weekly cisplatin. In FIGS. 11A and 11B, in the graph on the left, the EC-18-treated group (active) is shown on the right in each time period, and the placebo shown on the left for each time period.

### IL-1β Level Comparison Between EC-18 and Placebo Groups in Total.

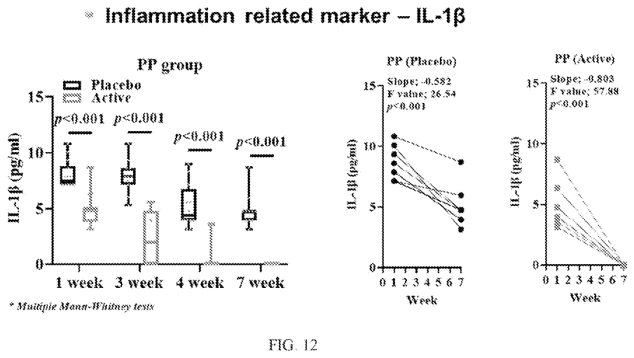

The IL-1β concentration in the EC-18 treated group was significantly lower than the placebo group throughout seven weeks treatment period. The concentration of IL-1β was decreased even under the detection limit at the 4th and 7th weeks of treatment. As shown in FIG. 12, the IL-1β concentration in the EC-18 treated group was significantly lower than the placebo group throughout seven weeks treatment period. In FIG. 12, in the graph on the left, the EC-18-treated group (active) is shown on the right in each time period, and the placebo shown on the left for each time period.

IL-1β Comparison Between EC-18 and Placebo Groups with SOM or without SOM

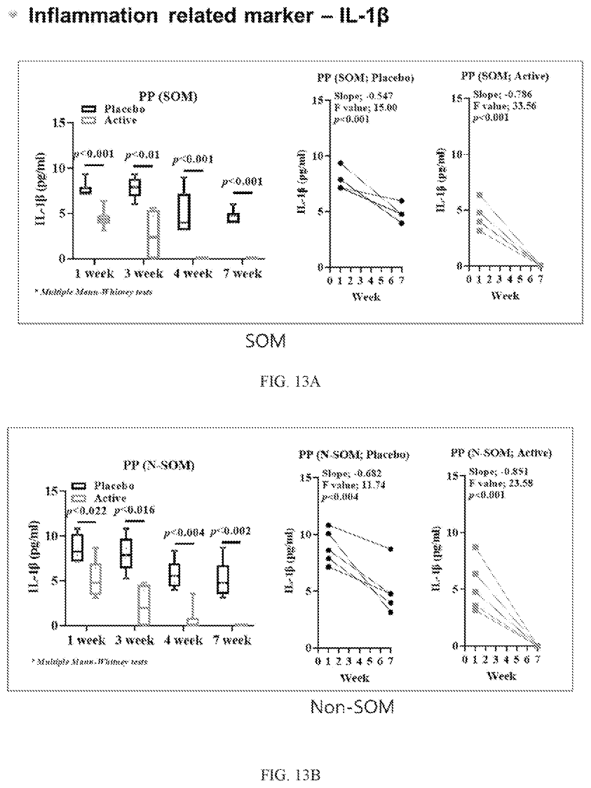

The IL-1β concentration in the EC-18 treated group was significantly lower compared with the placebo group regardless of the incidence of SOM. As shown in FIGS. 13A-13B, the IL-1β concentration in the EC-18 treated group was significantly lower compared with the placebo group regardless of the incidence of SOM. In FIGS. 13A and 13B, in the graph on the left, the EC-18-treated group (active) is shown on the right in each time period, and the placebo shown on the left for each time period.

IL-1β Level Comparison Between EC-18 and Placebo Groups with HPV+/HPV−

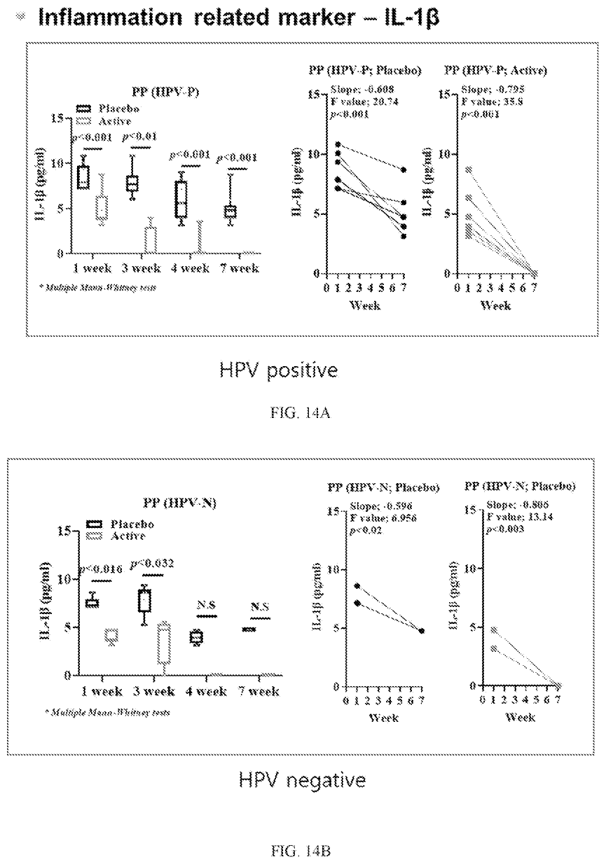

The IL-1β concentration in the EC-18 treated group was significantly lower compared with the placebo group regardless of HPV infection state. As shown in FIGS. 14A-14B, the IL-1β concentration in the EC-18 treated group was significantly lower compared with the placebo group regardless of HPV infection state. In FIGS, 14A and 14B, in the graph on the left, the EC-18-treated group (active) is shown on the right in each time period, and the placebo shown on the left for each time period.

4. IL-1β Level Comparison Between EC-18 and Placebo Groups with Cisplatin Weekly or Cisplatin Tri-Weekly

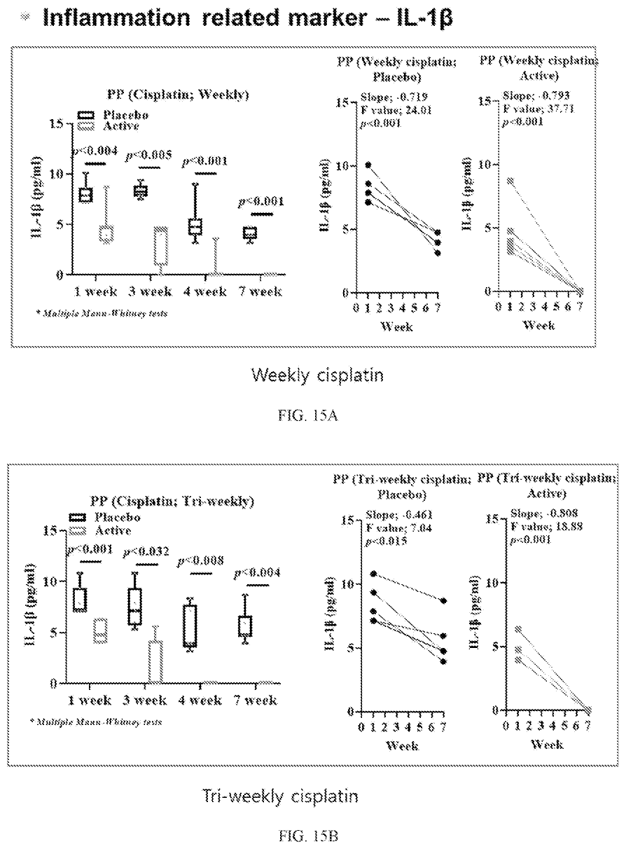

The IL-1β concentration in the EC-18 treated group was significantly lower than in the placebo group regardless of the weekly or tri-weekly cisplatin treatment. As shown in FIGS. 15A-15B, the IL-1β concentration in the EC-18 treated group was significantly lower than in the placebo group regardless of the weekly or tri-weekly cisplatin treatment. In FIGS. 15A and 15B, in the graph on the left, the EC-18-treated group (active) is shown on the right in each time period, and the placebo shown on the left for each time period.

All documents mentioned herein are fully incorporated herein by reference.

1. A method for treating a human subject having mucositis,
comprising administering to the human subject an effective amount of EC-18, thereby treating the mucositis.

2-6. (canceled)

7. The method of claim 1 wherein the human subject has been diagnosed with head or neck cancer.

8. The method of claim 1 wherein the human patient is receiving treatment for cancer.

9. The method of claim 8 wherein the human subject is receiving cisplatin therapy.

10-18. (canceled)

19. The method of claim 1 wherein the human subject has been diagnosed as suffering from head or neck cancer.

20. The method of claim 1 wherein the human subject has been diagnosed as suffering from one or more symptoms of mucositis.

21. The method of claim 1 wherein the human subject has been diagnosed as suffering from oral ulceration.

22. The method of claim 1 wherein EC-18 is co-administered with one or more distinct agents for treatment of mucositis.

23. The method of claim 1 wherein EC-18 is co-administered with one or more of palifermin; pentoxifylline including with vitamin E; chlorhexidine gluconate (including oral rinse); hyaluronate including sodium hyaluronate; a superoxide dismutase mimetic such as superoxide dismutase mimetic M40403; dusquetide or other innate defense regulator.

24. A kit comprising:
(a) EC-18;
(b) instructions for using EC-18 to treat mucositis in a human subject; and
(c) one or more of palifermin; pentoxifylline including with vitamin E; chlorhexidine gluconate; hyaluronate; a superoxide dismutase mimetic3; dusquetide or an innate defense regulator

25-34. (canceled)

35. A method for treating a human subject is 1) identified as suffering mucositis and 2) identified as HPV positive,
comprising administering to the human subject an effective amount of EC-18, thereby treating or preventing the mucositis.

36. The method of claim 35 further comprising 1) identifying the subject as suffering from mucositis and being BPV positive and 2) administering to the identified subject the EC-18.

37-44. (canceled)

45. The method of claim 35 wherein the human subject has been diagnosed with head or neck cancer.

46. The method of claim 35 wherein the human subject is receiving treatment for cancer.

47. The method of claim 47 wherein the human subject is receiving cisplatin therapy.

48-53. (canceled)

54. The method of claim 1 wherein the human subject has oral mucositis.

55. The method of clam 1 wherein the human subject has mucositis associated with chemotherapy.

56. The method of claim 1 wherein the human subject has head and neck cancer.

57. The method of claim 56 wherein the human subject has HPV.

58. The method of claim 57 wherein the human subject is receiving cisplatin therapy.
